(19) United States
(12) Patent Application Publication
(10) Pub. No.: US 2015/0297140 A1
(43) Pub. Date: Oct. 22, 2015

(54) USER STRESS DETECTION AND MITIGATION

(71) Applicant: Microsoft Corporation, Redmond, WA (US)

(72) Inventors: Javier Hernandez, Cambridge, MA (US); Asta Roseway, Bellevue, WA (US); Mary Czerwinski, Kirkland, WA (US); Pablo Enrique Paredes Castro, San Leandro, CA (US); Daniel Choi, Bellevue, WA (US)

(73) Assignee: Microsoft Corporation, Redmond, WA (US)

(21) Appl. No.: 14/257,950

(22) Filed: Apr. 21, 2014

(51) Int. Cl. A61B 5/00; G01N 21/25; A61B 19/00; A61B 5/16; A61B 5/053; G01N 27/22; G01L 5/00

(52) U.S. Cl. CPC A61B 5/6897; G01N 27/22; G01N 21/25; G01L 5/00; A61B 5/165; A61B 5/7435; A61B 5/053; A61B 5/7282; A61B 19/46; A61B 5/7264; A61B 2562/0214; A61B 2562/0247; A61B 2019/465

(57) ABSTRACT

Embodiments for responding to user stress are provided. In one embodiment, a method performed on a computing device comprises detecting a contact area size on a mouse in communication with the computing device, assessing a user stress level based on the contact area size, and outputting an indication of the user stress level.

### BACKGROUND

Chronic stress may lead to a wide variety of negative health outcomes. Typical methods used to determine the stress level of a user tend to rely on obtrusive querying mechanisms, such as self-reporting or assessing various physiological signals.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

A method performed on a computing device for responding to user stress comprises detecting a contact area size on a mouse in communication with the computing device, assessing a user stress level based on the contact area size, and outputting an indication of the user stress level.

### BRIEF DESCRIPTION OF THE DRAWINGS

FIG. 1 schematically shows a stress detection and mitigation environment.

FIG. 2 is a flow chart illustrating a method for detecting and mitigating user stress.

FIG. 3 shows a schematic of a non-limiting computing system.

### DETAILED DESCRIPTION

The repeated triggering of the stress reflex during daily activity may result in chronic stress, leading to a large array of adverse health conditions such as depression, hypertension, and various forms of cardiovascular diseases. In order to mitigate user stress, the stress level of a user of a computing device may be detected in an unobtrusive and continuous, semi-continuous, or periodic manner so that user stress can be assessed without creating additional stress on the user. To detect user stress in an unobtrusive manner, sensor readings from one or more input devices of the computing device may be monitored. For example, the pressure applied to the keys of a pressure-sensitive keyboard and/or the pressure, contact area, etc., of input applied to a capacitive mouse may be monitored to sense the manifestations of stress in the user. If the user appears to be operating under a relatively high amount of stress, one or more actions may be taken to attempt to mitigate the user's stress, such as delaying notifications and/or computer updates displayed to the user, or by outputting a notification to the user indicating his or her stress level.

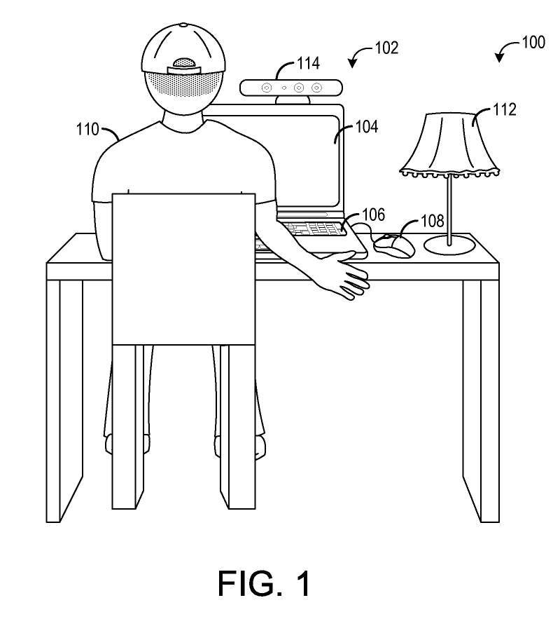

FIG. 1 shows a schematic diagram of a user stress detection and mitigation environment 100. Environment 100 includes a computing device 102 operated by a user 110. Computing device 102 may include any suitable device, such as a desktop computer, laptop, tablet, mobile computing device (e.g., smart phone), or other suitable device. Computing device 102 includes a logic machine and a data holding machine in communication with one or more input, display, and/or peripheral devices. For example, as illustrated in FIG. 1, computing device 102 may be operatively coupled to a peripheral display device 104, peripheral keyboard 106, peripheral mouse 108, and peripheral feedback device (herein illustrated as a lamp 112). The data holding machine stores instructions that are executable, for example, to receive and interpret inputs from the input device(s) and to send output to the display device 104. Example hardware configurations are described in more detail below.

In some embodiments, display device 104, keyboard 106, and mouse 108 may each be a separate component in communication with computing device 102. In other embodiments, one or more of display device 104, keyboard 106, and mouse 108 may be integrated with computing device 102 (e.g., as a tablet computer or smart phone).

Keyboard 106 may be a pressure-sensitive keyboard configured to measure a relative amount of pressure applied by the user with each keystroke. Accordingly, keyboard 106 may include one or more pressure sensors or other pressure-detecting mechanisms. Mouse 108 may be a capacitive mouse including, for example, a capacitive grid configured to measure the capacitance caused by user manipulation of the mouse (e.g., detect the location and/or pressure of touch input to the mouse). Further, display device 104 may be a touch sensitive display device configured to detect touch input to the display device 104 via one or more image, capacitive, or other sensors. The above-described examples are non-limiting, however, and other types of keyboards, mice, display devices, and/or other peripherals are within the scope of this disclosure.

During operation of computing device 102, user 110 may apply input to one or more of the keyboard 106, mouse 108, and display device 104. Various physical parameters of the user input may be detected and/or measured based on output from the sensors of the input devices. For example, the pressure, speed, regularity, and/or accuracy of the keystrokes made by the user to the keyboard may be monitored based on output from the pressure sensors of the pressure-sensitive keyboard. In another example, the pressure, hand-mouse contact area, and/or mouse-surface contact area of the manipulation of the mouse by the user may be measured by the capacitive sensors of the mouse. In a still further example, the pressure, speed, contact area, and/or accuracy of user touch input (e.g., swipes) to the display device may be detected.

Other types of user input physical parameters may also be monitored. For example, computing device 102 may include a sensor subsystem 114 including one or more image sensors, microphones, etc., configured to capture user posture, gestures, and/or voice input. If the user consents, the posture, gestures, and/or voice input may be interpreted by the computing device to determine a relative stress level of the user, for example an increased amount of gesturing or the use of strong language (e.g., expletives) may indicate a high level of stress. Further, user stress may be determined based on recognition of facial features associated with stress by information captured by the sensor subsystem 114. Example facial features associated with stress may include furrowed eyebrows, pursed lips, clenched jaw, flared nostrils, skin color (e.g., increased or decreased blood flow and/or heart rate may be captured by a thermal camera by comparing forehead and nose colors or heat maps).

Still further mechanisms may be used to detect user stress. Anything that can be physically manipulated on a device, including knobs, dials, and buttons, may be monitored to determine user input pressure, frequency of interaction, etc., to determine stress. Additionally, pressure sensitive pens may be monitored (e.g., how hard the user is pushing down or gripping the pen). Other examples of detected user stress may include determining how a user uses apps, frequency of app switching, use of apps in different contexts such as phone, mobile devices, computers, xbox, etc.

The measured physical parameters of the user input may be monitored to determine a relative stress level of the user. For example, when the user is operating with a high level of stress, he or she may type faster, depress the keyboard keys with greater pressure, make more typographical errors (detected by increased use of the backspace or delete key, for example), apply more pressure to the mouse, grip the mouse with more fingers (e.g., manipulate the mouse with a greater hand-mouse contact area), etc., than when the user is operating with a lower level of stress. In some embodiments, the various physical parameters of the user input may be considered individually to determine user stress level, while in other embodiments the different physical parameters may be considered collectively, e.g., multiple different physical parameters of the various user input mechanisms may be assessed to determine user stress. Further, the physical parameters of the different user inputs may be correlated to each other to determine the level of user stress.

User stress level as a function of the sensor readings from the input device or devices (e.g., the physical characteristics of the user input) may be determined in a suitable manner. In one example, the physical characteristics of the user input may be monitored over time to determine an average value for each physical characteristic of each input device for a given user. If a subsequent user input differs from the average, a change in user stress level may be determined. For example, the average pressure and speed at which the user depresses the keyboard keys may be determined over a given time period (e.g., one day, one week, one month, etc.). If, during a subsequent input to the keyboard, the pressure or speed of the keystrokes is greater than the average, it may be determined the user is experiencing an increase in stress.

The relative level of the user stress may correspond to the degree by which the input physical characteristic differs from the determined average. For example, if the keystroke pressure is more than one standard deviation greater than the average, it may be determined that the user is experiencing a medium level of stress, while if the keystroke pressure is more than two standard deviations greater than the average, it may be determined that the user is experiencing a high level of stress. Other mechanisms of correlating user stress to the physical characteristics of the user input are possible, such as a learning mode where the physical characteristics of the user input (e.g., keystroke pressure, mouse surface contact area) are mapped to various tasks assumed to create different user stress levels (e.g., browsing the Internet versus preparing a report for work).

Further, the type of stress a user is experiencing may be determined. Different types of stress may elicit different types of physical responses, and thus may be associated with different responses to the detected stress. Example types of stress include cognitive load, chronic stress, heightened arousal, remembering past memories, physical stress, fear, and danger. Additionally, some types of stress may be assumed to be desired or expected, depending on the context of the detected stress. For example, heightened arousal may be acceptable in certain contexts, such as games where stress can be helpful.

If it is detected that the user is experiencing a relatively high level of stress, one or more actions may be taken to assist the user in mitigating his or her stress. For example, the environment in which the user is working may be made more soothing by adjusting the lighting, sound volume, or other environmental conditions. In other examples, the user may be notified in an unobtrusive manner that his or her stress level has increased. For example, lamp 112 may be adjusted to output a warmer light color to create a more soothing environment, and/or the light output by the lamp 112 may be adjusted to subtly notify the user that his or her stress level has increased. Other examples of mechanisms for notifying the user include adjusting a system tray icon, adjusting a color of the keyboard, adjusting a color of the display device, providing feedback via clothing (e.g. clothing that hugs the user and/or clothing that provides haptic feedback that mimics a tap or tap on the shoulder), outputting an auditory notification, etc. By notifying the user of his or her stress level, the user may take measures to reduce his or stress, such as taking a walk, meditating, etc. In some embodiments, a person other than the user could additionally or alternatively be notified of the user's stress level, such as a family member or the user's social network.

Further, in some embodiments, actions may be taken to prevent the user from experiencing further stress. Example stress-preventing actions may include delaying or dispensing with push notifications, updates, messages or other forms of non-vital communication, or other computing device tasks not related to the tasks the user is currently undergoing.

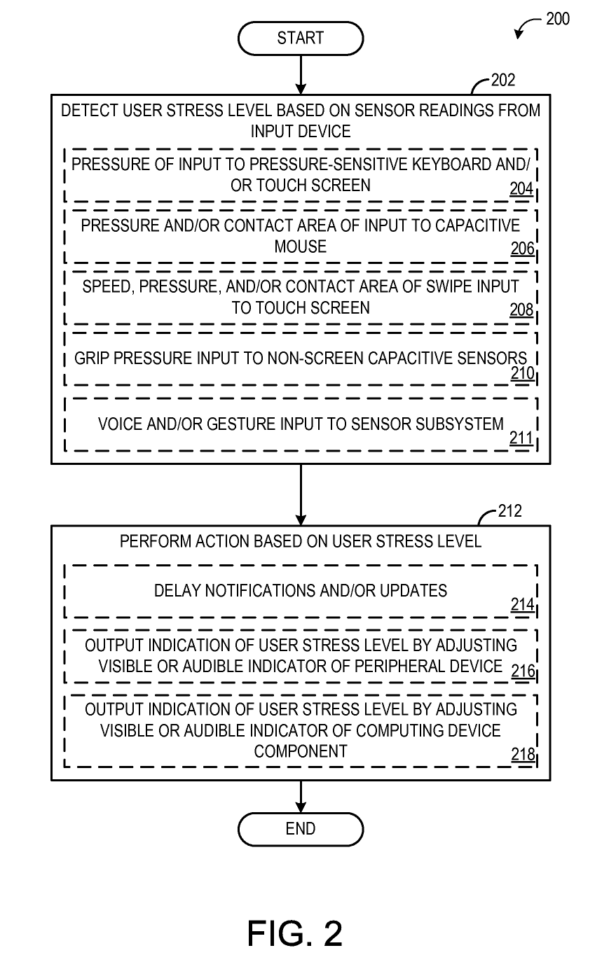

FIG. 2 is a flow chart illustrating a method 200 for detecting and mitigating user stress. Method 200 may be carried out by a computing device, such as computing device 102, coupled to or in communication with one or more input and/or peripheral devices, such as display device 104, keyboard 106, mouse 108, and lamp 112.

At 202, method 200 includes detecting user stress level based on sensor readings from a user input device. The sensor readings may indicate the physical properties of the user input to the user input device. When a user is experiencing stress (caused, for example, by a pressing deadline, unpleasant email, or other computing or non-computing task), various physiological changes may manifest, including pupil dilation, deeper breathing, increased heart rate, and increased muscle tension. These physiological changes may result in changes to the manner in which the user interacts with objects in his or her physical space, such as computer input devices. For example, a user may type more vigorously or handle a computer mouse more actively.

Thus, as indicated at 204, the pressure of user input applied to a pressure sensitive keyboard and/or touch screen may be monitored. As explained above, the keyboard may include pressure sensors or other pressure-detecting devices. If the pressure applied to the keyboard by the user increases above a threshold (e.g., above an average pressure determined for that user), it may be determined the user is experiencing increased stress. Further, as indicated at 206, the pressure and/or contact area of input to a capacitive mouse may be monitored. The capacitive grid of the mouse may allow the location of each touch input (e.g., each finger) to the mouse to be detected. In one example, the inclusion of a larger touch contact area, such as the inclusion of the thumb and four fingers as opposed to three fingers, on the mouse may indicate the user is experiencing stress. Further still, as indicated at 208, the speed, pressure, and/or contact area of a swipe input to a touch-sensitive device may be monitored. Additionally, some hand-held computing devices, such as tablets or smart phones, may include non-screen capacitive sensors on the side or sides of the device that are configured to detect the relative hand grip pressure the user applies to the device. As indicated at 210, the hand grip pressure applied to the device, as sensed by the non-screen capacitive sensors, may be monitored to determine the user stress level. As indicated at 211, a further mechanism for determining user stress may include voice and/or gesture input as detected by a sensor subsystem including one or more image sensors and/or microphones.

If the user is not experiencing an increased or relatively high level of stress, the characteristics of the environment and settings of the computing device may continue without adjustment. However, the physiological changes associated with increased stress, described above, may result in degraded health and/or well-being of the user, particularly if the user experiences them over a relatively long period of time. As such, it may be desirable for a user to be notified when he or she is undergoing stress, so that the user can attempt to mitigate the stress. Accordingly, if the physical characteristics of the user input indicate the user is experiencing stress, method 200 includes performing an action based on the user stress level at 212.

The action or actions taken in response to the user stress level may be any suitable action that helps mitigate the stress or indicate to the user that he or she is manifesting symptoms of stress. The actions may include, but are not limited to, delaying scheduled notifications and/or updates to the computing device, as indicated at 214. In some examples, the scheduled updates and/or notifications may be delayed or dispensed with only if they are not applicable to the task the user is currently performing.

Other actions that may be performed responsive to increased user stress include notifying the user of his or her stress level. This may include, as indicated at 216, adjusting a visible or audible indicator on a peripheral device, such as adjusting the color of light output by a lamp, adjusting the volume of music output by one or more speakers, etc. This may also include, as indicated at 218, adjusting a visible or audible indicator of the computing device, such as adjusting the color of light emitted by the keyboard, adjusting the color of light emitted by at least a portion the background screen of the display device, adjusting an icon displayed on the display device, or other indicators. If the color of light output by the lamp, screen color, or other environmental factor is adjusted, not only may the user be notified of his or her stress level, but the environment may also be made more soothing to attempt to mitigate the user stress.

The adjustment to the peripheral and/or computing device may be made in correspondence to the level of user stress, such that as user stress increases, the adjustment changes. For example, if the user is experiencing a relatively low level of increased stress, the peripheral device may be configured to output a particular color of light (e.g., green). If the user stress increases to a relatively higher level of stress, the peripheral device may be configured to output a different color of light (e.g., blue).

In some embodiments, the methods and processes described herein may be tied to a computing system of one or more computing devices. In particular, such methods and processes may be implemented as a computer-application program or service, an application-programming interface (API), a library, and/or other computer-program product.

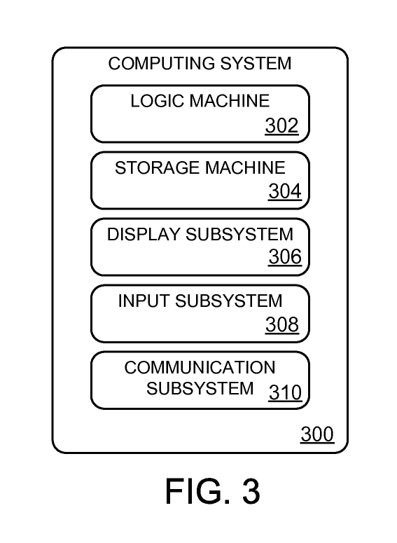

FIG. 3 schematically shows a non-limiting embodiment of a computing system 300 that can enact one or more of the methods and processes described above. Computing device 102 is one non-limiting example of computing system 300. Computing system 300 is shown in simplified form. Computing system 300 may take the form of one or more personal computers, server computers, tablet computers, home-entertainment computers, network computing devices, gaming devices, mobile computing devices, mobile communication devices (e.g., smart phone), and/or other computing devices.

Computing system 300 includes a logic machine 302 and a storage machine 304. Computing system 300 may optionally include a display subsystem 306, input subsystem 308, communication subsystem 310, and/or other components not shown in FIG. 3.

Logic machine 302 includes one or more physical devices configured to execute instructions. For example, the logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic machine may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of the logic machine optionally may be distributed among two or more separate devices, which may be remotely located and/or configured for coordinated processing. Aspects of the logic machine may be virtualized and executed by remotely accessible, networked computing devices configured in a cloud-computing configuration.

Storage machine 304 includes one or more physical devices configured to hold instructions executable by the logic machine to implement the methods and processes described herein. When such methods and processes are implemented, the state of storage machine 304 may be transformed—e.g., to hold different data.

Storage machine 304 may include removable and/or built-in devices. Storage machine 304 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage machine 304 may include volatile, nonvolatile, dynamic, static, read/write, read-only, random-access, sequential-access, location-addressable, file-addressable, and/or content-addressable devices.

It will be appreciated that storage machine 304 includes one or more physical devices. However, aspects of the instructions described herein alternatively may be propagated by a communication medium (e.g., an electromagnetic signal, an optical signal, etc.) that is not held by a physical device for a finite duration.

Aspects of logic machine 302 and storage machine 304 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 300 implemented to perform a particular function. In some cases, a module, program, or engine may be instantiated via logic machine 302 executing instructions held by storage machine 304. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display subsystem 306 may be used to present a visual representation of data held by storage machine 304. This visual representation may take the form of a graphical user interface (GUI). As the herein described methods and processes change the data held by the storage machine, and thus transform the state of the storage machine, the state of display subsystem 306 may likewise be transformed to visually represent changes in the underlying data. Display subsystem 306 may include one or more display devices utilizing virtually any type of technology. Such display devices may be combined with logic machine 302 and/or storage machine 304 in a shared enclosure, or such display devices may be peripheral display devices.

When included, input subsystem 308 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication subsystem 310 may be configured to communicatively couple computing system 300 with one or more other computing devices. Communication subsystem 310 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 300 to send and/or receive messages to and/or from other devices via a network such as the Internet.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. On a computing device, a method for responding to user stress, comprising:
detecting a contact area size on a mouse in communication with the computing device;
assessing a user stress level based on the contact area size; and
outputting an indication of the user stress level.

2. The method of claim 1, further comprising detecting the contact area size based on output from one or more capacitive sensors of the mouse.

3. The method of claim 1, wherein outputting an indication of the user stress level comprises adjusting a color of light emitted by a peripheral device in communication with the computing device in correspondence to the user stress level.

4. The method of claim 1, wherein outputting an indication of the user stress level comprises adjusting a color of light emitted by a component of the computing device in correspondence to the user stress level.

5. The method of claim 1, wherein outputting an indication of the user stress level comprises adjusting an icon displayed on a display of the computing device in correspondence to the user stress level.

6. On a computing device, a method for responding to user stress, comprising:
detecting a user stress level based on sensor readings from an input device; and
performing an action based on the user stress level.

7. The method of claim 6, wherein detecting a user stress level comprises detecting a user stress level based on a pressure of user input to a keyboard.

8. The method of claim 6, wherein detecting a user stress level comprises detecting a user stress level based on a contact area size of user input to a mouse.

9. The method of claim 8, further comprising detecting the contact area size based on output from one or more capacitive sensors of the mouse.

10. The method of claim 8, wherein detecting a user stress level based on a contact area size of user input to a mouse comprises detecting a contact area size of user input to a mouse relative to an average contact area size.

11. The method of claim 6, wherein detecting a user stress level comprises detecting a user stress level based on a user hand grip pressure applied to the computing device.

12. The method of claim 11, further comprising detecting the user hand grip pressure based on output from one or more non-screen capacitive sensors of the computing device.

13. The method of claim 6, wherein performing an action based on the user stress level comprises delaying notifications scheduled to be displayed on a display of the computing device.

14. The method of claim 6, wherein performing an action based on the user stress level comprises outputting an indication of the user stress level.

15. The method of claim 14, wherein outputting an indication of the user stress level comprises adjusting a color of light emitted by a peripheral device in communication with the computing device in correspondence to the user stress level.

16. The method of claim 14, wherein outputting an indication of the user stress level comprises adjusting a color of light emitted by a component of the computing device in correspondence to the user stress level.

17. The method of claim 14, wherein outputting an indication of the user stress level comprises adjusting an icon displayed on a display of the computing device in correspondence to the user stress level.

18. A computing device comprising a storage machine holding instructions executable by a logic machine to:
detect a user stress level based on sensor readings from an input device in communication with the computing device; and
adjust a color of light emitted by at least a portion of one or more of a peripheral device, a keyboard, and a display of the computing device in response to the user stress level.

19. The computing device of claim 18, wherein the instructions are executable to detect the user stress level based on a pressure of the user input applied to the input device.

20. The computing device of claim 18, wherein the instructions are executable to detect the user stress level based on a contact area size of the user input applied to the input device.
